(19) United States
(12) Patent Application Publication
(10) Pub. No.: US 2026/0097219 A1
(43) Pub. Date: Apr. 9, 2026

(54) ESCALATING DEFIBRILLATION THERAPIES BASED ON ARRHYTHMIA BURDENS

(71) Applicant: Stryker Corporation, Portage, MI (US)

(72) Inventors: Robert G. Walker, Seattle, WA (US); Tyson G. Taylor, Bothell, WA (US); Rose Tingwei Yin, Bellevue, WA (US); Fred W. Chapman, Newcastle, WA (US)

(73) Assignee: Stryker Corporation, Portage, MI (US)

(21) Appl. No.: 19/352,383

(22) Filed: Oct. 7, 2025

Related U.S. Application Data
(60) Provisional application No. 63/705,489, filed on Oct. 9, 2024.

(51) Int. Cl. A61N 1/39

(52) U.S. Cl. CPC A61N 1/3904

(57) ABSTRACT

An example method includes determining, by analyzing an electrocardiogram (ECG) of a subject, a time period between administration of a first electrical shock to the subject and a subsequent initiation of a shockable arrhythmia of the subject. The example method further includes predicting that a multi-shock therapy is indicated by analyzing the time period; and in response to predicting that the multi-shock therapy is indicated, causing administration of the multi-shock therapy to the subject, the multi-shock therapy including a second electrical shock and a third electrical shock.

### CROSS-REFERENCE TO RELATED APPLICATION

This application claims the priority of U.S. Provisional Application No. 63/705,489, which was filed on Oct. 9, 2024 and is incorporated by reference herein in its entirety.

### BACKGROUND

Particular heart arrhythmias, such as ventricular fibrillation (VF) and pulseless ventricular tachycardia (VT), are deadly if untreated. An individual with one of these arrhythmias can be treated by administering an electrical shock to the individual's heart. This treatment is known as defibrillation. The arrhythmias that are treatable by defibrillation are known as shockable arrhythmias.

Unfortunately, many instances of shockable arrhythmias are resistant to conventional defibrillation therapies. For instance, an individual is determined to have refractory VF if they have VF that continues through the administration of an electrical shock. Recently, double sequential defibrillation (DSD) (also referred to as “double sequential external defibrillation” or “DSED”) has been proposed as an alternative to single-shock defibrillation. A DSD therapy, for instance, can be administered by discharging two electrical shocks to an individual's heart, rather than one. Researchers have suggested that DSD administration can increase survivability of refractory VF. Cheskes et al., 387 N. Engl. J. Med. 1947 (2022).

### BRIEF DESCRIPTION OF THE DRAWINGS

FIG. 1 illustrates an environment for escalating a defibrillation therapy based on an arrhythmia burden.

FIG. 2 illustrates an example rescue event in which an escalated defibrillation therapy is indicated and applied to a subject.

FIG. 3 illustrates an example process for causing administration of a multi-shock therapy based on a length of a time period between the administration of a single-shock therapy and the initiation of a shockable arrhythmia.

FIG. 4 illustrates an example process for causing administration of a multi-shock therapy based on an amount of time that an ECG of a subject is indicative of a shockable arrhythmia.

FIG. 5 illustrates an example of an external defibrillator configured to perform various functions described herein.

FIGS. 6A and 6B illustrate examples of environments and timing related to administering a multi-shock therapy. FIG. 6A shows an environment configured to administer the multi-shock therapy. FIG. 6B shows a timing relationship of multiple shocks administered in the multi-shock therapy.

### DETAILED DESCRIPTION

A multi-shock therapy, in some cases, may be indicated when a subject is experiencing refractory VF. For example, a multi-shock therapy (e.g., a DSD therapy) may successfully treat VF that has resisted treatments including multiple single-shock therapies. However, if the multi-shock therapy is only administered after the administration of multiple single-shock therapies, the subject may remain in VF for an extended period of time that can result in a severe hypoxic injury. This danger can be particularly acute if the subject was in VF for an extended period of time prior to the administration of the single-shock therapies. Accordingly, it may be preferred to administer an escalated defibrillation therapy, such as a multi-shock therapy, to the subject prior to attempting to administer multiple single-shock therapies.

Various implementations described herein relate to techniques for determining whether to escalate a defibrillation therapy based on an arrhythmia burden. In various cases, the burden of a shockable arrhythmia, such as VF or pulseless VT, on the health of a subject is related to the amount of time that the subject experiences the shockable arrhythmia. For instance, the longer the subject remains in VF during an acute medical emergency, the greater the risk of hypoxic injury to the subject. In some cases, a score representing the burden is calculated based on the amount of time that the subject experiences the shockable arrhythmia. If the amount of time, or the score, exceeds a threshold, then a defibrillation therapy may be automatically escalated, regardless of the number of defibrillation shocks previously administered to the subject. Accordingly, escalated defibrillation therapies, such as a multi-shock therapy or a single electrical shock having an escalated energy level (e.g., an energy level that is greater than an energy level of an electrical shock that was previously administered), can be administered to a subject in need thereof without a significant delay. In various implementations of the present disclosure, the risk of the subject for developing a hypoxic injury can be significantly reduced.

Implementations of the present disclosure will now be described with reference to the accompanying figures.

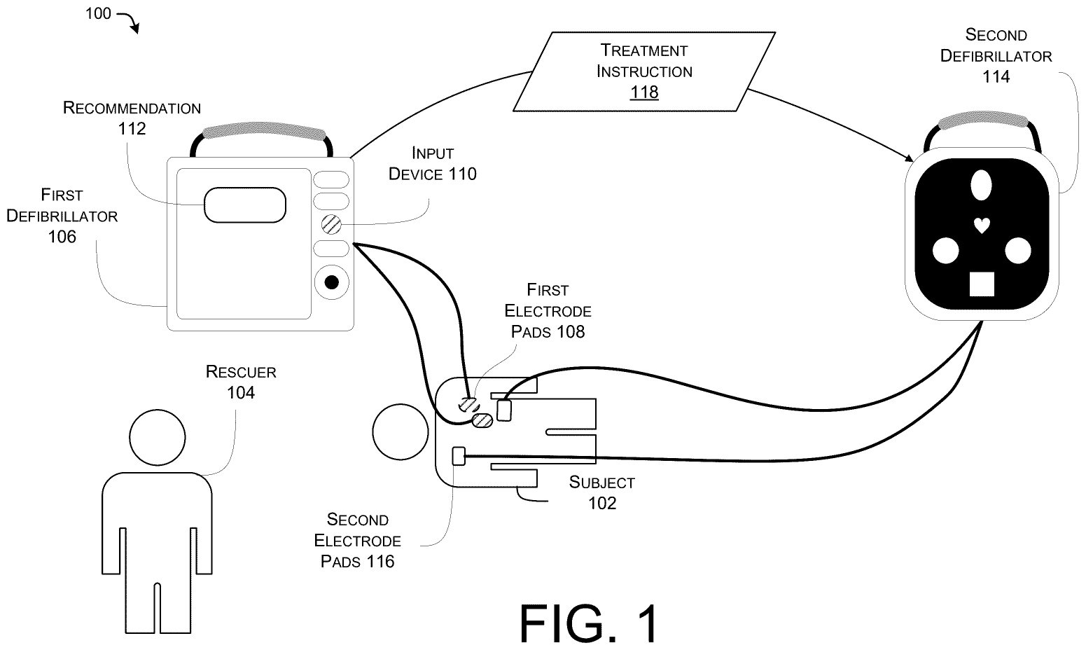

FIG. 1 illustrates an environment 100 for escalating a defibrillation therapy based on an arrhythmia burden. In some cases, the environment 100 is a clinical environment, such as within a hospital, medical clinic, hospice, or other environment designed to provide medical care. In some examples, the environment 100 is a non-clinical environment. For example, the environment 100 may be a public space, such as an airport terminal, school, office building, home, or other environment in which individuals can experience sudden medical emergencies.

A subject 102 has a suspected medical condition. In some cases, the subject 102 presents with one or more symptoms of the medical condition, such as dizziness, nausea, loss-of-consciousness, seizure, or some other indication that the subject 102 has the medical condition. In various cases, the medical condition is, or is associated with, an arrhythmia. As used herein, the term “arrhythmia,” and its equivalents, can refer to an irregular heart rhythm.

In various cases, a rescuer 104 is deployed to the side of the subject 102 in response to the medical condition. In some examples, the rescuer 104 is a nurse, a physician, a physician's assistant, or some other person with specific clinical knowledge. In some cases, the rescuer 104 is an emergency medical services (EMS) professional. In some examples, the rescuer 104 has been deployed to the environment 100 in response to a report that the subject 102 is experiencing the medical condition. For instance, the rescuer 104 has been deployed to the environment 100 in response to a call indicating that the subject 102 has suddenly collapsed in the environment 100.

To monitor the medical condition of the subject 102, the rescuer 104 utilizes a first defibrillator 106. The first defibrillator 106, for instance, is a monitor-defibrillator. According to some cases, the first defibrillator 106 is a wearable defibrillator. In some cases, the first defibrillator 106 is a portable medical device. For example, the rescuer 104 carried the first defibrillator 106 to the environment 100 from a vehicle (e.g., an ambulance or other emergency response vehicle). The first defibrillator 106 is configured to detect one or more physiological parameters of the subject 102 via one or more accessory devices. Examples of physiological parameters include electrocardiogram (ECG), electroencephalogram (EEG), blood flow (e.g., instantaneous blood velocity, volumetric blood flow, etc.), pulse rate, heart rate, blood oxygenation (e.g., regional oxygenation, pulse oximetry, oxygen saturation, etc.), airway parameters (e.g., flow rate of air in the airway, a partial pressure of oxygen and/or carbon dioxide in the airway, airway pressure, etc.), blood pressure (e.g., pulse pressure, systolic blood pressure, diastolic blood pressure, mean arterial blood pressure, etc.), physiological sounds (e.g., heart sounds, breath sounds, etc.), and the like. The accessory device(s) include one or more sensors, such as one or more electrodes, an ultrasound transducer (e.g., configured to detect physiological structures and/or fluid movement using the Doppler effect), an oximetry sensor, a flow sensor, an oxygen sensor, a carbon dioxide sensor, a blood pressure sensor (e.g., a blood pressure cuff, catheter sensor, etc.), a microphone, an accelerometer, a gyroscope, and the like.

In particular cases, the first defibrillator 106 is electrically coupled with first electrode pads 108 disposed on the skin of the subject 102. For instance, the first electrode pads 108 are adhered to the skin on the chest and/or back of the subject 102. The first electrode pads 108, for example, extend along a first vector that intersects the heart of the subject 102. The first vector, for example, is an electrical path that passes through the body of the subject 102.

The first defibrillator 106 is configured to detect an ECG of the subject 102 using the first electrode pads 108. The ECG, for instance, is representative of an electrical signal output by the heart of the subject 102 over time. The ECG is detected as a voltage between electrodes within the first electrode pads 108, for instance. Although the first electrode pads 108 are illustrated as having a particular position on the chest of the subject 102, implementations are not so limited. The position of the first electrode pads 108 may be anywhere on the torso, provided that at least a portion of the electrical path between the electrode pads 108 extends through the heart of the subject 102.

In various cases, the medical condition of the subject 102 is in danger of worsening. For example, the subject 102 may have an arrhythmia that is resistant to treatments, such as defibrillation. In various cases, the subject 102 has a shockable arrhythmia, such as ventricular fibrillation (VF) or pulseless ventricular tachycardia (VT). The first defibrillator 106 is configured to detect the shockable arrhythmia by analyzing the ECG (e.g., a single lead, 3-lead, 12-lead, or any combination thereof), in various cases, In some cases, the first defibrillator 106 determines that the subject 102 has the shockable arrhythmia by additionally analyzing one or more additional physiological parameters of the subject 102, such as at least one of EEG, transthoracic impedance, blood flow (e.g., instantaneous blood velocity, volumetric blood flow, etc.), pulse rate, heart rate, blood oxygenation (e.g., regional oxygenation, pulse oximetry, oxygen saturation, etc.), airway parameters (e.g., flow rate of air in the airway, a partial pressure of oxygen and/or carbon dioxide in the airway, airway pressure, etc.), blood pressure (e.g., pulse pressure, systolic blood pressure, diastolic blood pressure, mean arterial blood pressure, etc.), physiological sounds (e.g., heart sounds, breath sounds, etc.), or any combination thereof.

In various implementations of the present disclosure, the heart of the subject 102 is unable to effectively pump blood through the body of the subject 102 when the heart is exhibiting the shockable arrhythmia. Accordingly, the subject 102 may experience a hypoxic injury whose severity is proportional to the amount of time that the heart is exhibiting the 1 shockable arrhythmia and/or other arrhythmias that prevent the heart from spontaneously circulating blood throughout the body of the subject 102. In some cases, the hypoxic injury is associated with the brain of the subject 102. If sufficiently severe, the hypoxic injury can cause permanent damage to the subject 102, or even death.

In various instances, the first defibrillator 106 is configured to treat the shockable arrhythmia by outputting an electrical shock to the first electrode pads 108. This process can be referred to as “defibrillation.” However, in some cases, the shockable arrhythmia continues after the electrical shock is administered. VF that continues, without even temporarily abating, after the administration of an electrical shock is referred to as “refractory VF.” In some examples, the shockable arrhythmia temporarily abates after the administration of the electrical shock, but eventually recurs. VF that temporarily resolves after administration of an electrical shock is referred to as “recurrent VF.” Thus, the hypoxic injury of the subject 102 can increase in severity even after one or more electrical shocks are administered to the subject 102.

In various implementations of the present disclosure, it may be preferred to escalate a defibrillation therapy based on the risk of the subject 102 for developing a severe hypoxic injury. In some implementations, an escalated defibrillation therapy includes administration of a multi-shock therapy. For example, rather than applying a single electrical shock to the subject 102, the multi-shock therapy includes administering multiple electrical shocks to the subject 102. The multiple electrical shocks may be administered without further monitoring (e.g., an independent evaluation of the ECG of the subject 102) by the first defibrillator 106 between the multiple shocks. In some cases, the electrical shocks are temporally overlapping. The defibrillation therapy can be escalated in other ways, such as by increasing an energy level (e.g., increasing the energy level of an electrical shock to greater than 200 J, 360 J, or greater than a previously applied electrical shock) or duration of one or more electrical shocks administered to the subject 102. In some cases, the defibrillation therapy is escalated by changing a vector of one or more electrical shocks administered to the subject 102.

In various cases, an escalated defibrillation therapy has some drawbacks. For example, some escalated defibrillation therapies are more likely to cause burns to the subject 102, or cause other types of harm. However, in some cases, the escalated defibrillation therapy is more likely to successfully treat (e.g., permanently resolve) the shockable arrhythmia than a non-escalated defibrillation therapy. In cases in which the subject 102 is at risk for developing a serious hypoxic injury, it may be preferred to escalate the defibrillation therapy despite the potential drawbacks of escalation.

For example, the first defibrillator 106 determines whether to escalate a defibrillation therapy based on an analysis of an amount of time that the ECG of the subject 102 is indicative of the shockable arrhythmia and/or any other heart rhythm associated with insufficient circulation (e.g., asystole, bradycardia, etc.). In various cases, if the amount of time that the ECG of the subject 102 is indicative of the shockable arrhythmia is greater than a threshold, the first defibrillator 106 determines that an escalated defibrillation therapy is indicated.

In some cases, the first defibrillator 106 determines whether to escalate a defibrillation therapy based on an analysis of an amount of time that the ECG of the subject 102 is indicative of a heart rhythm that is not the shockable arrhythmia (e.g., normal sinus rhythm, etc.). For example, if the amount of time that the ECG of the subject 102 is indicative of the heart rhythm that is not the shockable arrhythmia is lower than a threshold, then the first defibrillator 106 determines that the escalated defibrillation therapy is indicated.

According to some examples, the first defibrillator 106 calculates a metric, referred to as a “score,” in order to determine whether the escalated defibrillation therapy is indicated. When the shockable arrhythmia is VF, the score can be referred to as a “VF score.” The score, for instance, is representative of a burden of one or more shockable arrhythmias on the body of the subject 102. This burden, for example, may be associated with the resistance of the shockable arrhythmia to treatment and/or the likelihood that the subject 102 will experience an injury (e.g., a hypoxic injury) due to the shockable arrhythmia.

In various implementations, the score is calculated based on the amount of time that the ECG of the subject is indicative of the shockable arrhythmia and/or any other heart rhythm associated with insufficient circulation (e.g., asystole, bradycardia, etc.). In some cases, the score is calculated based on the amount of time that the ECG of the subject is indicative of a heart rhythm that is not the shockable arrhythmia. For instance, Equation 1 is a calculation of the score as a fraction of time that the ECG is indicative of the shockable arrhythmia:

$$s = \frac{{t}_{s}}{{t}_{t}} \qquad ( 1 )$$

where s is the score, tₛ is an amount of time that the ECG is indicative of the shockable arrhythmia, and tₜ is the total amount of time represented by the ECG. Notably, in some cases, the ECG indicates multiple, distinct time periods in which the heart of the subject 102 is experiencing the shockable arrhythmia. Accordingly, tₛ may represent a sum of the lengths of these distinct time periods. In some cases, the first defibrillator 106 outputs one or more electrical shocks to the subject 102 during the total amount of time represented by the ECG. In some cases, the time periods during which the electrical shock(s) are administered are added to the total amount of time represented by tₛ.

In some cases, the score is a quotient representing the amount of time that the ECG of the subject is indicative of the shockable arrhythmia divided by the amount of time that the ECG of the subject is indicative of a heart rhythm that is not the shockable arrhythmia. For example, Equation 2 represents an alternate calculation of the score:

$$s = \frac{{t}_{s}}{{t}_{n}} \qquad ( 2 )$$

wherein tₙ represents an amount of time in the ECG that is not indicative of the shockable arrhythmia.

In some implementations, the score is further dependent on one or more additional factors. For example, the score may be calculated based on an amplitude and/or amplitude spectrum area (AMSA) of the ECG. For example, the score can be calculated using the following Equation 3:

$$s = {s}_{0} f ( a ) \qquad ( 3 )$$

wherein s₀ is the score calculated according to Equation 1 or 2 and f(a) is a function of the amplitude and/or AMSA of the ECG. In various cases, the amplitude and/or AMSA of the ECG is inversely proportional to the score.

According to some cases, the score is calculated based on a physiological parameter of the subject 102 that is indicative of the delivery of oxygen via the circulation of blood in the subject 102. Examples of these parameters include, for instance, blood oxygenation (e.g., pulse oxygenation), blood pressure (e.g., non-invasive blood pressure), blood flow (e.g., volumetric flow rate through at least one blood vessel), and an amount of carbon dioxide (CO₂) in the airway of the subject 102 (e.g., a partial pressure of CO₂ in the airway of the subject 102). For example, the score may be calculated using the following Equation 4:

$$s = {s}_{1} f ( p ) \qquad ( 4 )$$

wherein s₁ is the score calculated according to Equation 1, 2, or 3 and f(p) is a function of the physiological parameter indicative of the delivery of oxygen via the circulation of blood in the subject 102. In some cases, the parameter is inversely proportional to the score.

In some implementations, the score is calculated based on whether one or more parameters of the subject 102, including the ECG of the subject 102, are indicative of a condition. For instance, the score is calculated based on the following Equation 5:

$$s = {s}_{2} {f}_{1} ( c ) + {f}_{2} ( c ) \qquad ( 5 )$$

wherein s₂ is the score calculated according to Equation 1, 2, 3, or 4 f₁(c) is a first function of the presence (e.g., certainty or probability) of a detected condition of the subject 102, and f₂ (c) is a second function of the presence (e.g., certainty or probability, a number of detected events, a length of time in which the detected events are present, etc.) of a detected condition of the subject 102.

In some examples, the score is calculated based on characteristics of the subject 102 when the subject 102 is in cardiac arrest. For example, the score is calculated based on the ECG and/or condition of the subject 102 during one or more time intervals in which the subject 102 is in cardiac arrest. In some cases, the first defibrillator 106 is configured to infer that the subject 102 is in cardiac arrest based on detecting that chest compressions are being administered to the subject 102. For instance, the first defibrillator 106 is configured to detect the administration of chest compressions by identifying patterns in a transthoracic impedance of the subject 102 that are indicative of chest compressions. In some examples, the first defibrillator 106 determines that the subject 102 is receiving chest compressions based on a communication signal from a mechanical chest compression device that is administering the chest compressions, by analyzing a pressure administered to the chest of the subject 102, or any combination thereof.

According to some cases, the presence of spontaneous circulation (of blood) is another example of a condition that is relevant to the score. In some cases, a total amount of time that blood is circulating through the body of the subject 102 is used to calculate the score. In some cases, the first defibrillator 106 detects circulating blood (e.g., a return of spontaneous circulation (ROSC)) in the subject 102. For example, the first defibrillator 106 infers that the subject 102 has spontaneous circulation by analyzing one or more parameters indicative of blood circulation (e.g., blood pressure, blood oxygenation, blood flow parameters, or any combination thereof) during a time period in which the subject 102 is not receiving chest compressions. Other information relevant to the determination of spontaneous circulation include a time at which chest compressions are initiated, a time at which chest compressions are halted, the receipt of a 12-lead ECG after a period of chest compressions is detected, and the like.

For example, the score is calculated based on whether the ECG is indicative of a condition, such as coarse VF or fine VF. Coarse VF, in various cases, can be identified by determining that an AMSA or amplitude (e.g., peak amplitude, mean amplitude, etc.) of an ECG indicative of VF is above a threshold. In contrast, fine VF can be identified by determining that an AMSA or amplitude (e.g., peak amplitude, mean amplitude, etc.) of an ECG indicative of VF is below the threshold.

In some examples, the score is calculated based on whether the ECG is indicative of another type of condition, such as ST elevation myocardial infarction (STEMI), occlusion myocardial infarction (OMI), ST elevation, ST depression, excessive premature ventricular contractions (PVCs) (e.g., more than a threshold number of PVCs in a time interval and/or more than a threshold number of consecutive PVCs), insufficient PVCs (e.g., less than a threshold number of PVCs in a time interval and/or less than a threshold number of consecutive PVCs), or any combination thereof. In various cases, STEMI and/or OMI can be predicted based, at least in part, on the ECG. According to some cases, the first defibrillator 106 predicts that the subject has a STEMI by determining that an ST segment of at least one lead the ECG is elevated above a baseline (e.g., a level of a TP segment). OMI, for instance, can be predicted based on determining that an ST segment of at least one lead in the ECG is elevated, identifying acute T-waves, identifying terminal QRS distortion, identifying that an amplitude of one or more QRS complexes is below a threshold, or a combination thereof.

The first defibrillator 106, for instance, detects one or more PVCs of the subject 102 by analyzing the ECG. A PVC is a type of heartbeat that is initiated by ventricular tissue, as opposed to a sinus node, atrial tissue, or the AV node of the heart. In various cases, a PVC omits a p-wave, a t-wave of the PVC is larger than a t-wave of a QRS complex, a polarity of the t-wave is opposite to the polarity of a t-wave of a QRS complex, a longer QT interval than that of non-PVC QRS complexes, QRS complexes having greater than a threshold duration, or a combination thereof. One or more of these characteristics can be identified in the ECG of the subject 102, for instance. In various cases, three or more consecutive PVCs is classified as VT.

One or more of the conditions described above can be identified based, at least in part, on non-ECG parameters. For example, various arrhythmias and other heart-relevant conditions are evidenced by light-headedness (e.g., identifying a blood flow, blood oxygenation, or blood pressure below a threshold), shortness-of-breath (e.g., identifying a respiratory rate above a threshold), abnormal heart sounds, or any combination thereof.

In various implementations of the present disclosure, the first defibrillator 106 is configured to determine whether the escalated defibrillation therapy is indicated by comparing the score to one or more thresholds. For example, the first defibrillator 106 may conclude that the escalated defibrillation therapy is indicated by determining that the score is lower than a first threshold and/or by determining that the score is greater than a second threshold. In some cases, the first defibrillator 106 is further configured to determine whether the escalated defibrillation therapy is indicated based on an availability of one or more devices configured to administer the escalated defibrillation therapy. For instance, the first defibrillator 106 may determine whether another defibrillator is connected to the subject 102 or whether an accessory device configured to output at least a portion of the escalated defibrillation therapy is connected to the subject 102. In some cases, the first defibrillator 106 outputs a prompt to connect one or more devices that can administer the escalated defibrillation therapy to the subject 102. In some cases, more than one escalated defibrillation therapy is possible. In various cases, the first defibrillator 106 identifies that a particular escalated defibrillation therapy is indicated based on determining that one or more devices configured to administer the particular escalated defibrillation therapy are connected to the subject 102 (and, optionally, determining that one or more devices configured to administer another escalated defibrillation therapy are unavailable).

In some examples, the first defibrillator 106 outputs a recommendation 112 to administer the escalated therapy to the subject 102. The first defibrillator 106, for example, outputs the recommendation 112 in response to determining that the escalated defibrillation therapy is indicated. Although FIG. 1 illustrates that the recommendation 112 is output on a display of the first defibrillator 106, implementations are not so limited. For example, in some cases, the first defibrillator 106 is configured to output the recommendation 112 as an audible signal. Based on the recommendation 112, for instance, the rescuer 104 may apply the escalated defibrillation therapy. For example, the rescuer 104 may cause the first defibrillator 106 to output the escalated defibrillation therapy by activating an input device 110 of the first defibrillator 106. In some cases, the input device 110 includes a button and/or touch sensor that detects pressing and/or touching of the input device 110 by the rescuer 104.

According to some cases, the first defibrillator 106 causes the escalated defibrillation therapy to be administered to the subject 102 in response to determining that the escalated defibrillation therapy is indicated. For example, the first defibrillator 106 may output one or more electrical shocks to the first electrode pads 108, wherein the electrical shock(s) are at least a portion of the escalated defibrillation therapy.

According to some cases, the first defibrillator 106 outputs at least a portion of the electrical shocks via one or more capacitors. For example, the first defibrillator 106 charges the capacitor(s) using an on-board power source (e.g., a battery) and outputs one or more of the electrical shocks by discharging the capacitor(s). In some cases, the first defibrillator 106 outputs one of the electrical shocks by completely discharging a single capacitor. In some examples, the first defibrillator 106 outputs multiple electrical shocks by sequentially and partially discharging a single capacitor.

In some examples, the escalated defibrillation therapy is administered jointly by the first defibrillator 106 and a second defibrillator 114. The second defibrillator 114, in various cases, is electrically coupled to second electrode pads 116 disposed on skin of the subject 102. For example, the second electrode pads 116 are adhered to skin on the chest and/or back of the subject 102. For instance, the second electrode pads 116 are arranged along a second vector that is different than the first vector. Although not specifically illustrated in FIG. 1, in some cases, the second defibrillator 114 is electrically connected with the first electrode pads 108.

The second defibrillator 114 is a portable medical device, in some cases. In various examples, the second defibrillator 114 is an automated external defibrillator (AED). In some cases, the second defibrillator 114 is a monitor-defibrillator, a wearable defibrillator, or an implantable defibrillator. Although not illustrated in FIG. 1, in some examples, the second defibrillator 114 is integrated into a mechanical chest compression device that is configured to administer chest compressions to the subject 102. In some cases, the second defibrillator 114 is a defibrillator accessory without a standalone monitoring capability.

In the example of FIG. 1, the first defibrillator 106 coordinates administration of the escalated defibrillation therapy with the second defibrillator 114 by transmitting a treatment instruction 118 to the second defibrillator 114. The treatment instruction 118, for instance, includes one or more communication signals instructing the second defibrillator 114 to administer one or more of electrical shocks in the escalated defibrillation therapy. In some cases, the treatment instruction 118 indicates one or more times at which the electrical shock(s) are to be administered by the second defibrillator 114. According to some cases, the treatment instruction 118 causes the second defibrillator 114 to activate one or more protection circuits that prevent electrical shocks output by the first defibrillator 106 from being absorbed by the circuitry within the second defibrillator 114.

The second defibrillator 114 is configured to output one or more of the electrical shocks to the second electrode pads 116 in response to the treatment instruction 118. In some examples, the second defibrillator 114 is configured to output one or more of the electrical shocks to the first electrode pads 108 in response to the treatment instruction 118. The second defibrillator 114 includes one or more capacitors. For instance, the second defibrillator 114 is configured to output one or more of the electrical shocks by discharging the capacitor(s). In some cases, the second defibrillator 114 delivers one of the electrical shocks by fully discharging one of the capacitor(s). In some examples, the second defibrillator 114 outputs multiple instances of the electrical shocks by partially discharging the one of the capacitor(s). In some implementations, the first defibrillator 106 and the second defibrillator 114 output alternating electrical shocks in the sequence of external electrical shocks.

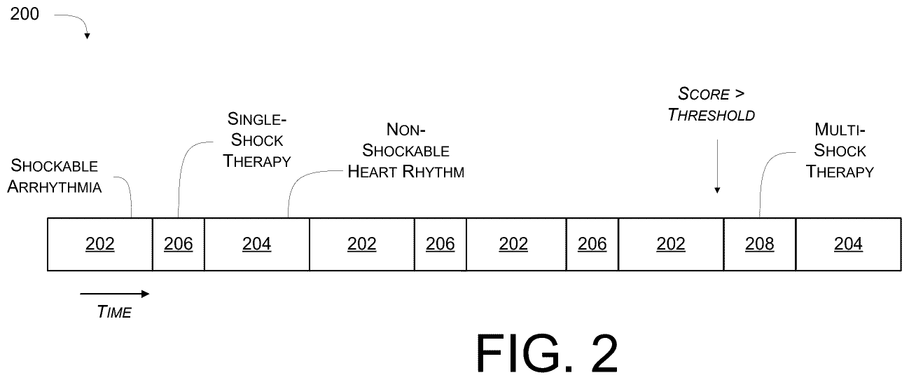

FIG. 2 illustrates an example rescue event 200 in which an escalated defibrillation therapy is indicated and applied to a subject. The rescue event 200 is divided into blocks representing different time intervals. In FIG. 2, time increases from left to right.

In various cases, the subject exhibits a shockable arrhythmia 202. Examples of the shockable arrhythmia 202 include VF and pulseless VT. In some cases, the presence of the shockable arrhythmia 202 is apparent by analyzing at least one segment of the ECG of the subject.

A single-shock therapy 206 is administered to the subject as a treatment for the shockable arrhythmia 202. In various cases, the single-shock therapy 206 is a multiphasic (e.g., biphasic) shock administered by a single defibrillator. For example, the single-shock therapy 206 is an external shock output to electrodes disposed on the skin of the subject.

In response to receiving the single-shock therapy 206, the subject exhibits a non-shockable heart rhythm 204 (e.g., a non-VF heart rhythm). Examples of the non-shockable heart rhythm 204 include a sinus rhythm (e.g., including QRS complexes), supraventricular tachycardia, one or more PVCs, pulseless electrical activity (PEA), asystole, or a combination thereof. The presence of the non-shockable heart rhythm indicates that the single-shock therapy 206 was at least temporarily successful.

Despite the initial effectiveness of the single-shock therapy 206, the subject re-enters the shockable arrhythmia 202. In various cases, the single-shock therapy 206 is applied to the subject a second time. However, the shockable arrhythmia 202 remains immediately after the single-shock therapy 206 is applied to the subject the second time. The shockable arrhythmia 202 continues even after the single-shock therapy 206 is administered to the subject a third time.

Eventually, the amount of time that the subject is in the shockable arrhythmia 202 is sufficiently high such that an escalated defibrillation therapy is eventually indicated. In some examples, a score is calculated that is dependent on the amount of time in the rescue event 200 that the subject is in the shockable arrhythmia 202, the amount of time in the rescue event 200 that the subject is receiving instances of the single-shock therapy 206, the amount of time in the rescue event 200 that the subject is exhibiting the non-shockable heart rhythm 204, or a combination thereof. In some cases, the score is further dependent on a type of condition indicated by the non-shockable heart rhythm, such as whether the subject is exhibiting characteristics of STEMI or OMI. In various cases, the escalated defibrillation therapy is indicated when the score is greater than a threshold. For example, the score may be greater than the threshold if the subject has exhibited the shockable arrhythmia 202 is greater than four, five, or six minutes; if the shockable arrhythmia 202 is exhibited more than a threshold percentage (e.g., in a range of 10 to 100%) of time between shocks; if a cumulative time of the shockable arrhythmia 202 is greater than a threshold percentage (e.g., in a range of 20 to 60%) of time within one or more chest compression intervals (e.g., a continuous interval in which chest compressions are administered to the subject); if the shockable arrhythmia 202 returns less than a threshold amount of time (e.g., in a range of 10 to 40 seconds) after an electrical shock has been administered; if the shockable arrhythmia 202 is present during a chest compression interval; or any combination thereof.

In various implementations, a multi-shock therapy 208 is administered to the subject when the score becomes greater than the threshold. The multi-shock therapy 208, for example, includes the administration of multiple electrical shocks to the subject. In some cases, the electrical shocks are temporally overlapping. For instance, the electrical shocks in the multi-shock therapy 208 are administered by one or more defibrillators.

In various cases, the subject exhibits the non-shockable heart rhythm 204 in response to receiving the multi-shock therapy 208. In some cases, the shockable arrhythmia 202 of the subject is resistant to the single-shock therapy 206, but the shockable arrhythmia 202 is susceptible to the multi-shock therapy 208. Accordingly, the escalation of the defibrillation therapy is warranted.

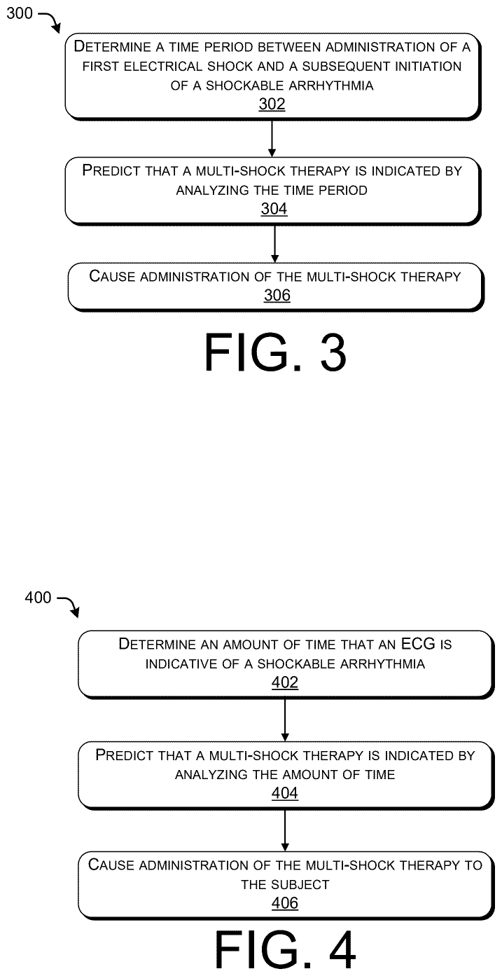

FIG. 3 illustrates an example process 300 for causing administration of a multi-shock therapy based on a length of a time period between the administration of a single-shock therapy and the initiation of a shockable arrhythmia. The process 300 is performed by an entity. The entity includes, for instance, at least one of a defibrillator (e.g., the first defibrillator 106 or the second defibrillator 114), a medical device, at least one processor, a computing device, or any combination thereof.

At 302, the entity determines a time period between administration of a first electrical shock and a subsequent initiation of a shockable arrhythmia. The shockable arrhythmia, for instance, includes VF or pulseless VT. For example, the entity determines a time at which the first electrical shock is administered (e.g., an end of the time period in which the first electrical shock is administered). In some cases, the entity further determines a time at which the ECG of the subject transitions into the shockable arrhythmia by analyzing the ECG. In some cases, the ECG is also indicative of the shockable arrhythmia during one or more time periods prior to the administration of the first electrical shock.

At 304, the entity predicts that a multi-shock therapy is indicated by analyzing the time period. In some cases, the entity predicts that the multi-shock therapy is indicated by determining that the time period is shorter than a threshold time period. In some cases, the entity determines a score indicating a burden of the shockable arrhythmia on the subject based, at least in part, on the time period. In some examples, the entity determines that the multi-shock therapy is indicated based on time periods in which the ECG is indicative of the shockable arrhythmia. For instance, if a sum of the time periods in which the ECG is indicative of the shockable arrhythmia is greater than a threshold, the entity may determine that the multi-shock therapy is indicated. In some examples, the entity determines the score based on an amount of time that the subject is exhibiting the shockable arrhythmia. In some cases, the score is further dependent on the amount of time that the ECG is indicative of the shockable arrhythmia, an amplitude of the ECG (e.g., a mean amplitude), an AMSA of the ECG, the presence of one or more characteristics associated with STEMI or OMI in the ECG, or a combination thereof. In various cases, the entity predicts that the multi-shock therapy is indicated by comparing the score to a threshold.

In some cases, the entity predicts that the multi-shock therapy is indicated by utilizing a trained machine learning model. The trained machine learning model, for instance, is defined by various parameters that are optimized based on training data including training features (e.g., scores, ECG segments, transthoracic impedances, indications of the administration of chest compressions (e.g., obtained from one or more mechanical chest compression devices administering the chest compressions), parameters detected during time periods indicating burdens of shockable arrhythmias derived from a population of individuals omitting the subject) as well as labels (e.g., indicating whether the multi-shock therapy is indicated for the individuals, or any combination thereof). In some cases, the ECG segments and/or transthoracic impedances are obtained before administration of electrical shocks, before recurrence of VF (and after administration of the electrical shocks), after recurrence of VF (and after administration of the electrical shocks, or any combination thereof. For example, the machine learning model is trained by optimizing the parameters in order to minimize a loss between the labels and an output of the machine learning model in response to receiving the training features. According to various cases, the entity may input the time period and/or score into the trained machine learning model and identify whether the multi-shock therapy is indicated based on an output of the machine learning model. Examples of suitable machine learning models, for instance, include decision trees, naïve Bayes classifiers, random forests, linear discriminant analysis (LDA) models, k-nearest neighbor (KNN) models, or any combination thereof.

At 306, the entity causes administration of the multi-shock therapy. The multi-shock therapy, for instance, includes administration of a second electrical shock and a third electrical shock to the subject. In some cases, the second electrical shock and the third electrical shock are administered along different vectors. In some examples, the second electrical shock and the third electrical shock are administered along the same vector. In various cases, the second electrical shock and the third electrical shock are administered by one or more defibrillators. The second electrical shock and the third electrical shock, in various cases, are temporally overlapping.

FIG. 4 illustrates an example process 400 for causing administration of a multi-shock therapy based on an amount of time that an ECG of a subject is indicative of a shockable arrhythmia. The process 400 is performed by an entity. The entity includes, for instance, at least one of a defibrillator (e.g., the first defibrillator 106 or the second defibrillator 114), a medical device, at least one processor, a computing device, or any combination thereof.

At 402, the entity determines an amount of time that the ECG is indicative of the shockable arrhythmia. The shockable arrhythmia, for instance, includes VF or pulseless VT. The shockable arrhythmia, for instance, is identified in an ECG of the subject. In some cases, the entity infers that the subject has the shockable arrhythmia during one or more time periods in which the subject has received an electrical shock.

In various implementations, the amount of time is represented by a fraction of time in the ECG that the shockable arrhythmia appears, a length of time in which the shockable arrhythmia appears, or a combination thereof. In some cases, the ECG indicates multiple events of the shockable arrhythmia, which may be separated by one or more events of non-shockable heart rhythms. The amount of time that the ECG is indicative of the shockable arrhythmia, for example, is calculated by adding the lengths of the multiple events together.

At 404, the entity predicts that the multi-shock therapy is indicated by analyzing the amount of time. In some cases, the entity predicts that the multi-shock therapy is indicated by determining that the amount of time is greater than a threshold. In some examples, the entity generates a score based on the amount of time and one or more additional factors (e.g., an AMSA of the ECG of the shockable arrhythmia, a mean amplitude of the ECG of the shockable arrhythmia, one or more characteristics associated with STEMI or OMI, or a combination thereof). The entity, for instance, predicts that the multi-shock therapy is indicated by comparing the score to a threshold.

At 406, the entity causes administration of the multi-shock therapy to the subject. In various implementations, the multi-shock therapy includes the administration of multiple electrical shocks to the subject. The electrical shocks, for instance, can be administered to electrodes disposed on the skin of the subject. In some cases, the electrical shocks are administered by multiple defibrillators. In some examples, the electrical shocks are administered by a single defibrillator. The electrical shocks, in some cases, are sequentially administered to the subject. In various cases, the electrical shocks are at least partially temporally overlapping.

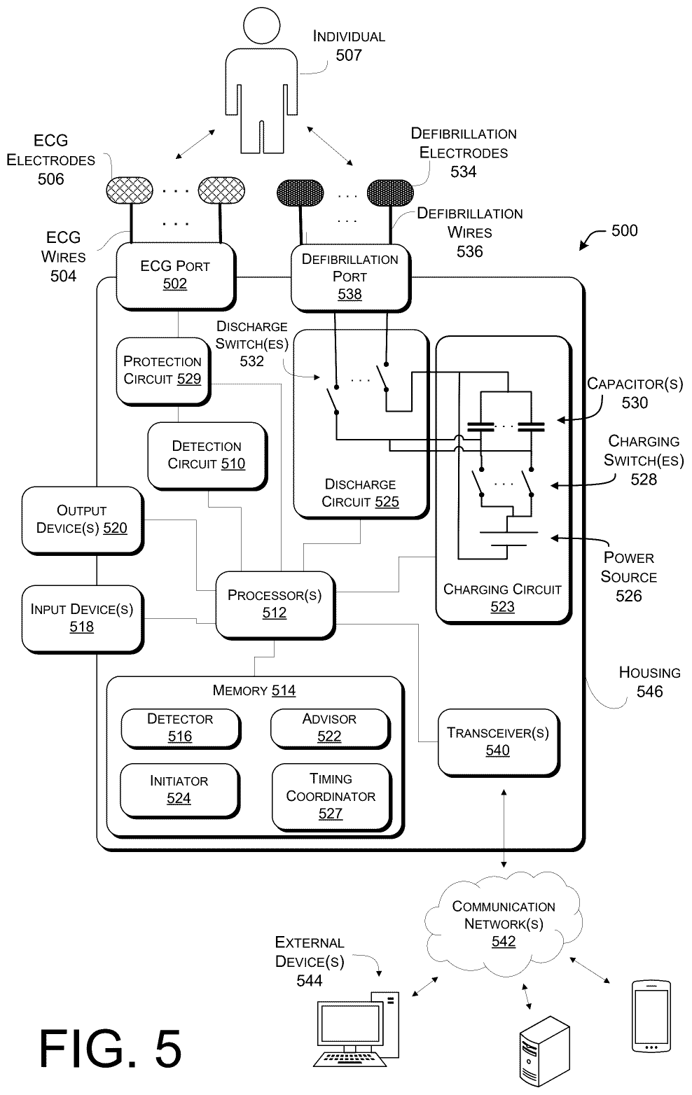

FIG. 5 illustrates an example of an external defibrillator 500 configured to perform various functions described herein. For example, the external defibrillator 500 is the first defibrillator 106 or secondary defibrillator 106 described above with reference to FIG. 1.

The external defibrillator 500 includes an electrocardiogram (ECG) port 502 connected to multiple ECG wires 504. In some cases, the ECG wires 504 are removeable from the ECG port 502. For instance, the ECG wires 504 are plugged into the ECG port 502 via connectors. The ECG wires 504 are connected to ECG electrodes 506, respectively. In various implementations, the ECG electrodes 506 are disposed on different locations on an individual 508. A detection circuit 510 (also referred to as a “measurement circuit”) is configured to detect relative voltages between the ECG electrodes 506. These voltages are indicative of the electrical activity of the heart of the individual 508.

In various implementations, the ECG electrodes 506 are in contact with the different locations on the skin of the individual 508. In some examples, a first one of the ECG electrodes 506 is placed on the skin between the heart and right arm of the individual 508, a second one of the ECG electrodes 506 is placed on the skin between the heart and left arm of the individual 508, and a third one of the ECG electrodes 506 is placed on the skin between the heart and a leg (either the left leg or the right leg) of the individual 508. In these examples, the detection circuit 510 is configured to measure the relative voltages between the first, second, and third ECG electrodes 506. Respective pairings of the ECG electrodes 506 are referred to as “leads,” and the voltages between the pairs of ECG electrodes 506 are known as “lead voltages.” In some examples, more than three ECG electrodes 506 are included, such that 5-lead or 12-lead ECG signals are detected by the detection circuit 510.

The detection circuit 510 includes at least one analog circuit, at least one digital circuit, or a combination thereof. The detection circuit 510 receives the analog electrical signals from the ECG electrodes 506, via the ECG port 502 and the ECG wires 504. In some cases, the detection circuit 510 includes one or more analog filters configured to filter noise and/or artifact from the electrical signals. The detection circuit 510 includes an analog-to-digital (ADC) in various examples. The detection circuit 510 generates a digital signal indicative of the analog electrical signals from the ECG electrodes 506. This digital signal can be referred to as an “ECG signal” or an “ECG.”

In some cases, the detection circuit 510 further detects an electrical impedance between at least one pair of the ECG electrodes 506. For example, the detection circuit 510 includes, or otherwise controls, a power source that applies a known voltage (or current) across a pair of the ECG electrodes 506 and detects a resultant current (or voltage) between the pair of the ECG electrodes 506. The impedance is generated based on the applied signal (voltage or current) and the resultant signal (current or voltage). In various cases, the impedance corresponds to respiration of the individual 508, chest compressions performed on the individual 508, and other physiological states of the individual 508. In various examples, the detection circuit 510 includes one or more analog filters configured to filter noise and/or artifact from the resultant signal. The detection circuit 510 generates a digital signal indicative of the impedance using an ADC. This digital signal can be referred to as an “impedance signal” or an “impedance.”

The detection circuit 510 provides the ECG signal and/or the impedance signal one or more processors 512 in the external defibrillator 500. In some implementations, the processor(s) 512 includes a central processing unit (CPU), a graphics processing unit (GPU), both CPU and GPU, or other processing unit or component known in the art.

The processor(s) 512 is operably connected to memory 514. In various implementations, the memory 514 is volatile (such as random access memory (RAM)), non-volatile (such as read only memory (ROM), flash memory, etc.) or some combination of the two. The memory 514 stores instructions that, when executed by the processor(s) 512, causes the processor(s) 512 to perform various operations. In various examples, the memory 514 stores methods, threads, processes, applications, objects, modules, any other sort of executable instruction, or a combination thereof. In some cases, the memory 514 stores files, databases, or a combination thereof. In some examples, the memory 514 includes, but is not limited to, RAM, ROM, electrically erasable programmable read-only memory (EEPROM), flash memory, or any other memory technology. In some examples, the memory 514 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 512 and/or the external defibrillator 500. In some cases, the memory 514 at least temporarily stores the ECG signal and/or the impedance signal.

In various examples, the memory 514 includes a detector 516, which causes the processor(s) 512 to determine, based on the ECG signal and/or the impedance signal, whether the individual 508 is exhibiting a particular heart rhythm. For instance, the processor(s) 512 determines whether the individual 508 is experiencing a shockable rhythm that is treatable by defibrillation. Examples of shockable rhythms include VF and ventricular tachycardia (V-Tach). In some examples, the processor(s) 512 determines whether any of a variety of different rhythms (e.g., asystole, sinus rhythm, atrial fibrillation (AF), etc.) are present in the ECG signal. In some implementations, the detector 516, when executed by the processor(s) 512, causes the processor(s) 512 to determine that the burden of a shockable arrhythmia on the individual 507 is sufficiently high such that an escalated defibrillation therapy (e.g., a multi-shock therapy) is warranted. For example, the detector 516 includes instructions for calculating a score and comparing the score to one or more thresholds.

The processor(s) 512 is operably connected to one or more input devices 518 and one or more output devices 520. Collectively, the input device(s) 518 and the output device(s) 520 function as an interface between a user and the defibrillator 500. The input device(s) 518 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 520 includes at least one of a display, a speaker, a haptic output device, a printer, or any combination thereof. In various examples, the processor(s) 512 causes a display among the input device(s) 518 to visually output a waveform of the ECG signal and/or the impedance signal. In some implementations, the input device(s) 518 includes one or more touch sensors, the output device(s) 520 includes a display screen, and the touch sensor(s) are integrated with the display screen. Thus, in some cases, the external defibrillator 500 includes a touchscreen configured to receive user input signal(s) and visually output physiological parameters, such as the ECG signal and/or the impedance signal.

In some examples, the memory 514 includes an advisor 522, which, when executed by the processor(s) 512, causes the processor(s) 512 to generate advice and/or control the output device(s) 520 to output the advice to a user (e.g., a rescuer). In some examples, the processor(s) 512 provides, or causes the output device(s) 520 to provide, an instruction to perform CPR on the individual 508. In some cases, the processor(s) 512 evaluates, based on the ECG signal, the impedance signal, or other physiological parameters, CPR being performed on the individual 508 and causes the output device(s) 520 to provide feedback about the CPR in the instruction. According to some examples, the processor(s) 512, upon identifying that a shockable rhythm is present in the ECG signal, causes the output device(s) 520 to output an instruction and/or recommendation to administer a defibrillation shock to the individual 508.

The memory 514 also includes an initiator 524 which, when executed by the processor(s) 512, causes the processor(s) 512 to control other elements of the external defibrillator 500 in order to administer a defibrillation shock to the individual 508. In some examples, the processor(s) 512 executing the initiator 524 selectively causes the administration of the defibrillation shock based on determining that the individual 508 is exhibiting the shockable rhythm and/or based on an input from a user (received, e.g., by the input device(s) 518. In some cases, the processor(s) 512 causes the defibrillation shock to be output at a particular time, which is determined by the processor(s) 512 based on the ECG signal and/or the impedance signal.

In various cases, the memory 514 further includes a timing coordinator 527 that, when executed by the processor(s) 512, causes the processor(s) 512 to identify a timing relationship of electrical shocks in a multi-shock therapy, as well as to cause the electrical shocks to be output at the timing relationship. In some examples, the electrical shocks are output by the defibrillator 500. In some cases, the processor(s) 512 coordinate timing of electrical shocks administered by both the defibrillator 500 and an additional defibrillator. For example, the processor(s) 512 generate a shock instruction that causes the additional defibrillator to output a secondary electrical shock at a future time. In some cases, the timing coordinator 527, when executed by the processor(s) 512, causes the processor(s) 512 to identify QRS complexes in the ECG and causes the electrical shocks to be synchronized with the QRS complexes. In some examples, the timing relationship is a predetermined timing relationship.

In some implementations, the processor(s) 512 selectively activate a protection circuit 529 in response to executing at least some instructions in the timing coordinator 527. For example, the processor(s) 512 connect the protection circuit 259 to a path between the ECG port 502 and the detection circuit 510 in response to activating a multi-shock mode. The protection circuit 259, for instance, includes one or more diodes that prevent current from being induced in the detection circuit 510 due to administration of an electrical shock to the individual 507 by another defibrillator coupled with the individual 507. In some cases, the processor(s) 512 deactivates the input device(s) 518 and/or the output device(s) 520 when the defibrillator 500 is in the multi-shock mode.

The processor(s) 512 is operably connected to a charging circuit 523 and a discharge circuit 525. In various implementations, the charging circuit 523 includes a power source 526, one or more charging switches 528, and one or more capacitors 530. The power source 526 includes, for instance, a battery. The processor(s) 512 initiates an electrical shock by causing the power source 526 to charge at least one capacitor among the capacitor(s) 530. For example, the processor(s) 512 activates at least one of the charging switch(es) 528 in the charging circuit 523 to complete a first circuit connecting the power source 526 and the capacitor to be charged. Then, the processor(s) 512 causes the discharge circuit 525 to discharge energy stored in the charged capacitor across a pair of defibrillation electrodes 534, which are in contact with the individual 508. For example, the processor(s) 512 deactivates the charging switch(es) 528 completing the first circuit between the capacitor(s) 530 and the power source 526 and activates one or more discharge switches 532 completing a second circuit connecting the charged capacitor 530 and at least a portion of the individual 508 disposed between defibrillation electrodes 534. In some cases, an electrical shock is administered to the individual 507 by partially discharging the capacitor(s) 530.

The energy is discharged from the defibrillation electrodes 534 in the form of an electrical shock. For example, the defibrillation electrodes 534 are connected to the skin of the individual 508 and located at positions on different sides of the heart of the individual 508, such that the electrical shock is applied across the heart of the individual 508. In some cases, the electrical shock has a multiphasic (e.g., biphasic) waveform. In some examples, a sequence of multiple electrical shocks can prevent the individual 507 from developing an arrhythmia, such as VF. The discharge switch(es) 532 are controlled by the processor(s) 512, for example. In various implementations, the defibrillation electrodes 534 are connected to defibrillation wires 536. The defibrillation wires 536 are connected to a defibrillation port 538, in implementations. According to various examples, the defibrillation wires 536 are removable from the defibrillation port 538. For example, the defibrillation wires 536 are plugged into the defibrillation port 538.

In various implementations, the processor(s) 512 is operably connected to one or more transceivers 540 that transmit and/or receive data over one or more communication networks 542. For example, the transceiver(s) 540 includes a network interface card (NIC), a network adapter, a local area network (LAN) adapter, or a physical, virtual, or logical address to connect to the various external devices and/or systems. In various examples, the transceiver(s) 540 includes any sort of wireless transceivers capable of engaging in wireless communication (e.g., radio frequency (RF) communication). For example, the communication network(s) 542 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 540 includes other wireless modems, such as a modem for engaging in WI-FI®, WIGIG®, WIMAX®, BLUETOOTH®, or infrared communication over the communication network(s) 542.

The defibrillator 500 is configured to transmit and/or receive data (e.g., ECG data, impedance data, data indicative of one or more detected heart rhythms of the individual 508, data indicative of one or more defibrillation shocks administered to the individual 508, etc.) with one or more external devices 544 via the communication network(s) 542. The external devices 544 include, for instance, mobile devices (e.g., mobile phones, smart watches, etc.), Internet of Things (IoT) devices, medical devices (e.g., a primary defibrillator or secondary defibrillator), computers (e.g., laptop devices, servers, etc.), or any other type of computing device configured to communicate over the communication network(s) 542. In some examples, the external device(s) 544 is located remotely from the defibrillator 500, such as at a remote clinical environment (e.g., a hospital). According to various implementations, the processor(s) 512 causes the transceiver(s) 540 to transmit data to the external device(s) 544. In some cases, the transceiver(s) 540 receives data from the external device(s) 544 and the transceiver(s) 540 provide the received data to the processor(s) 512 for further analysis. In some cases, the defibrillator 500 is configure to transmit or receive a shock instruction via the communication network(s) 542.

In various implementations, the external defibrillator 500 also includes a housing 546 that at least partially encloses other elements of the external defibrillator 500. For example, the housing 546 encloses the detection circuit 510, the processor(s) 512, the memory 514, the charging circuit 523, the transceiver(s) 540, or any combination thereof. In some cases, the input device(s) 518 and output device(s) 520 extend from an interior space at least partially surrounded by the housing 546 through a wall of the housing 546. In various examples, the housing 546 acts as a barrier to moisture, electrical interference, and/or dust, thereby protecting various components in the external defibrillator 500 from damage.

In some implementations, the external defibrillator 500 is an automated external defibrillator (AED) operated by an untrained user (e.g., a bystander, layperson, etc.) and can be operated in an automatic mode. In automatic mode, the processor(s) 512 automatically identifies a rhythm in the ECG signal, makes a decision whether to administer a defibrillation shock, charges the capacitor(s) 530, discharges the capacitor(s) 530, or any combination thereof. In some cases, the processor(s) 512 controls the output device(s) 520 to output (e.g., display) a simplified user interface to the untrained user. For example, the processor(s) 512 refrains from causing the output device(s) 520 to display a waveform of the ECG signal and/or the impedance signal to the untrained user, in order to simplify operation of the external defibrillator 500.

In some examples, the external defibrillator 500 is a monitor-defibrillator utilized by a trained user (e.g., a clinician, an emergency responder, etc.) and can be operated in a manual mode or the automatic mode. When the external defibrillator 500 operates in manual mode, the processor(s) 512 cause the output device(s) 520 to display a variety of information that may be relevant to the trained user, such as waveforms indicating the ECG data and/or impedance data, notifications about detected heart rhythms, and the like.

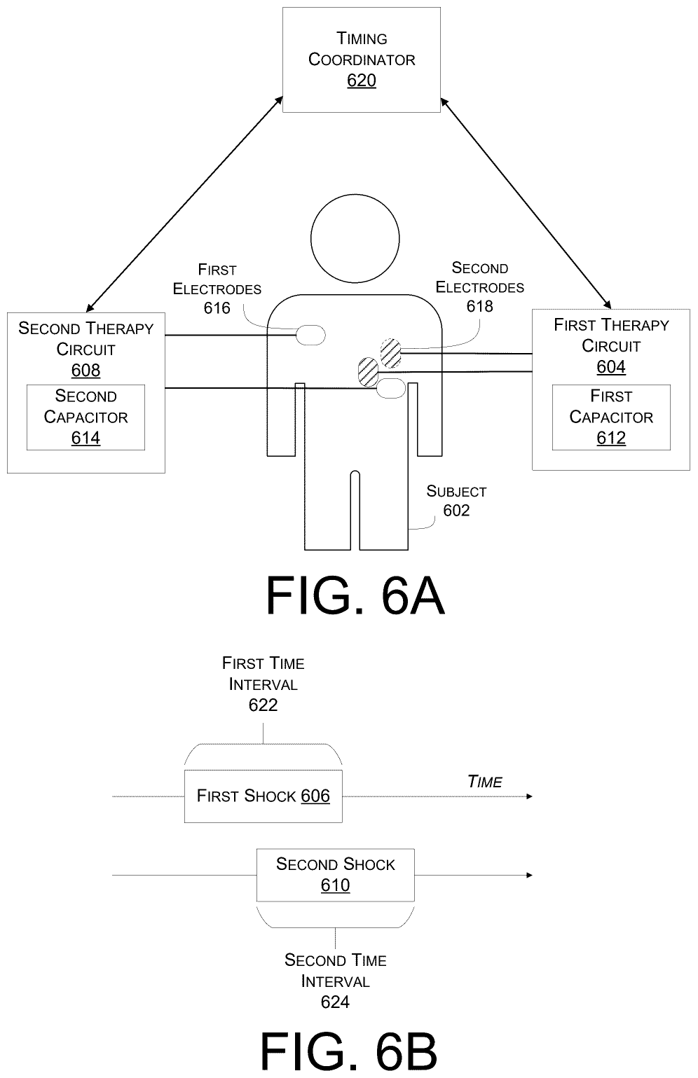

FIGS. 6A and 6B illustrate examples of environments and timing related to administering a multi-shock therapy. FIG. 6A shows an environment configured to administer the multi-shock therapy. FIG. 6B shows a timing relationship of multiple shocks administered in the multi-shock therapy.

In various cases, a subject 602 is predicted to have an arrhythmia that can be treated and/or prevented by the administration of a multi-shock therapy. In some cases, the multi-shock therapy is determined to be indicated when the subject 602 is determined to have a sufficiently high burden of a shockable arrhythmia (e.g., as indicated by a score associated with the shockable arrhythmia). Specifically, a first therapy circuit 604 is configured to output a first shock 606 to the subject 602 and a second therapy circuit 608 is configured to output a second shock 610 to the subject 602. In various cases, the first shock 606 and the second shock 610 temporally overlap in time, at least partially. For example, a start time of the second shock 610 occurs after the start time of the first shock 606, but the start time of the second shock 610 occurs before the end time of the first shock 606. In various cases, the first shock 606 is a biphasic shock and/or the second shock 610 is a biphasic shock. In some implementations, the first shock 606 is a monophasic shock and/or the second shock 610 is a biphasic shock. In some cases, the first shock 606 has a shorter duration and/or lower voltage amplitude than the second shock 610.

The first therapy circuit 604 outputs the first shock 606 by discharging a first capacitor 612. Similarly, the second therapy circuit 608 outputs the second shock 610 by discharging a second capacitor 614. In various implementations, one or more power sources are configured to charge the first capacitor 612 and/or the second capacitor 614 prior to discharge. In various cases, the first therapy circuit 604 includes a first H-bridge circuit including the first capacitor 612 and/or the second therapy circuit 608 includes a second H-bridge circuit including the second capacitor 614. The first H-bridge circuit and the second H-bridge circuit are configured to output the first shock 606 and the second shock 610 as biphasic shocks, for instance, via sequential activation of switches in the first H-bridge circuit and the second H-bridge circuit.

The first therapy circuit 604 is configured to output the first shock 606 to first electrodes 616. The second therapy circuit 608 is configured to output the second shock 610 to second electrodes 618. In various cases, the first electrodes 616 and/or the second electrodes 618 are disposed externally on the skin of the subject 602. For example, the first electrodes 616 and/or the second electrodes 618 are adhered to the skin of the subject 602. In various implementations, the first electrodes 616 and the second electrodes 618 are associated with different shock vectors. For instance, a first shock vector extends between the first electrodes 616 and a second shock vector extends between the second electrodes 618, wherein the first shock vector and the second shock vector are different. For example, the first shock vector may be an anterior-lateral position and the second shock vector may be an anterior-posterior position. In various implementations, the first shock vector and the second shock vector both extend through the heart of the subject 602. Although FIG. 6A illustrates the first electrodes 616 as being separate from the second electrodes 618, implementations are not so limited. For example, one electrode may be shared among the first electrodes 616 and the second electrodes 618.

The first therapy circuit 604 and the second therapy circuit 608 are distributed among one or more devices. In some cases, the first therapy circuit 604 is part of a first external defibrillator and the second therapy circuit 608 is part of a second external defibrillator. For example, the first external defibrillator and the second external defibrillator are both monitor-defibrillators, both AEDs, or a monitor-defibrillator and an AED. In some cases, the first therapy circuit 604 or the second therapy circuit 608 is integrated into an accessory device without monitoring capabilities, and which is solely designed to output electrical shocks upon receiving an input signal from a separate device. For example, the accessory device may lack, or be disconnected from, one or more sensors configured to identify one or more physiological parameters of the subject 602. In some cases, the accessory device lacks a display, speaker, or other user interface device. In some implementations, the first therapy circuit 604 and the second therapy circuit 608 are integrated into the same device, such as the same monitor-defibrillator.

Optionally, a timing coordinator 620 is configured to cause the first therapy circuit 604 to output the first shock 606 during a first time interval 622 and/or to cause the second therapy circuit 608 to output the second shock 610 during the second time interval 624. For example, the timing coordinator 620 outputs one or more signals (e.g., electrical signals, communication signals, etc.) to the first therapy circuit 604 and/or the second therapy circuit 608. Upon receiving the signal(s) from the timing coordinator 620, the first therapy circuit 604 may discharge the first capacitor 612 during the first time interval 622 and/or the second therapy circuit 608 may discharge the second capacitor 614 during the second time interval 624. The timing coordinator 620 can be implemented in hardware (e.g., a circuit), software (e.g., instructions executed by at least one processor), or a combination thereof. In some cases, the timing coordinator 620 is a standalone device. Examples of standalone timing devices that can serve as the timing coordinator 620 are described in U.S. Pat. No. 10,981,014, which is incorporated by reference herein in its entirety. In some examples, the timing coordinator 620 is integrated into the same device as the first therapy circuit 604 and/or the second therapy circuit 608.

Various timing relationships between the first time interval 622 and the second time interval 624 can be implemented according to various implementations of the present disclosure. In some cases, a delay between the start times (i.e., the leading edges) of the first time interval 622 and the second time interval 624 is in a range of 0 and 250 milliseconds (ms). In some cases, the delay between the start times of the first time interval 622 and the second time interval 624 is in a range of −250 and 0 ms. Although FIG. 6B illustrates the first time interval 622 and the second time interval 624 as having equivalent durations, implementations are not so limited. For example, the first time interval 622 may be longer or shorter than the second time interval 624. Various timing relationships are described in U.S. Pat. No. 10,702,701, which is incorporated by reference herein in its entirety.

In various cases, the timing coordinator 620 is configured to detect the first shock 606 and may cause the second therapy circuit 608 to output the second shock 610 in response. For example, the timing coordinator 620 may detect a signal indicative of the discharge of the first shock 606, and may output a signal that causes the second therapy circuit 608 to discharge the second shock 610. In some examples, the timing coordinator 620 is inductively coupled with the first therapy circuit 604 and/or the first electrodes 616, which enables the timing coordinator 620 to detect the discharge of the first shock 606. Various techniques for detecting the discharge of a first shock in order to cause the application of a second shock in multi-shock therapy are described in U.S. Pat. Nos. 10,625,088 and 10,632,320, which are incorporated by reference herein in their entirety.

### Example Clauses

1. A first external defibrillator, including: a measurement circuit configured to detect an electrocardiogram (ECG) of a subject; a treatment circuit configured to output a first electrical shock to the subject during a first time period and to output a second electrical shock to the subject during a second time period; and a processor configured to: determine, by analyzing the ECG of the subject: a time delay between administration of the first electrical shock and initiation of ventricular fibrillation (VF) in the ECG of the subject; and a fibrillation time period during which the ECG is indicative of the VF; determine a VF score as a function of the time delay and the fibrillation time period; predict that a multi-shock therapy is indicated by comparing the VF score to a threshold; and in response to predicting that the multi-shock therapy is indicated: outputting an instruction to administer a third electrical shock to the subject during a third time period, the third time period overlapping the second time period; and causing the treatment circuit to output the second electrical shock to the subject during the second time period.
2. The first external defibrillator of clause 1, wherein the processor is configured to determine the VF score further as a function of: an amplitude spectrum area (AMSA) of the VF; an amplitude of the VF; a presence of ST elevation in the ECG; and a presence of ST depression in the ECG.
3. The first external defibrillator of clause 1 or 2, further including: an output device configured to output the instruction to administer the third electrical shock to the subject during the third time period to a user; or a transceiver configured to output the instruction to administer the third electrical shock to the subject during the third time period to a second external defibrillator.
4. A method, including: determining, by analyzing an electrocardiogram (ECG) of a subject, a time period between administration of a first electrical shock to the subject and a subsequent initiation of a shockable arrhythmia of the subject, the shockable arrhythmia including ventricular fibrillation (VF) or ventricular tachycardia (VT); predicting that a multi-shock therapy is indicated by analyzing the time period; and in response to predicting that the multi-shock therapy is indicated, causing administration of the multi-shock therapy to the subject, the multi-shock therapy including a second electrical shock and a third electrical shock.
5. The method of clause 4, wherein determining, by analyzing the ECG of the subject, the time period between the administration of the first electrical shock to the subject and the subsequent initiation of the shockable arrhythmia of the subject includes: determining, by analyzing the ECG, a time at which the ECG of the subject transitions into the shockable arrhythmia.
6. The method of clause 4 or 5, wherein the ECG is indicative of the shockable arrhythmia during a time period before administration of the first electrical shock.
7. The method of any of clauses 4 to 6, wherein predicting that the multi-shock therapy is indicated by analyzing the time period includes: determining that the time period is less than a threshold time period.
8. The method of any of clauses 4 to 7, wherein predicting that the multi-shock therapy is indicated further includes: determining, by analyzing the ECG of the subject, a total amount of time in which the ECG is indicative of the shockable arrhythmia; and determining that the total amount of time in which the ECG is indicative of the shockable arrhythmia is above a threshold.
9. The method of any of clauses 4 to 8, wherein the shockable arrhythmia includes the VF and predicting that the multi-shock therapy is indicated further includes: determining that an amplitude of the VF exhibited by the ECG is above a threshold.
10. The method of any of clauses 4 to 9, wherein the shockable arrhythmia includes the VF predicting that the multi-shock therapy is indicated further includes: determining that an amplitude of the VF exhibited by the ECG is below a threshold.
11. The method of any of clauses 4 to 10, wherein predicting that the multi-shock therapy is indicated further includes: determining that the ECG is indicative of OMI or STEMI.
12. The method of any of clauses 4 to 11, wherein causing administration of the multi-shock therapy to the subject includes: causing administration of the first electrical shock along a first vector; and causing administration of the second electrical shock along the first vector or a second vector, the second vector being different than the first vector.
13. A medical device, including: a measurement circuit configured to detect an electrocardiogram (ECG) of a subject; and a processor configured to: determine, by analyzing the ECG of the subject, a time period between administration of a first electrical shock to the subject and a subsequent initiation of a shockable arrhythmia of the subject, the shockable arrhythmia including ventricular fibrillation (VF) or ventricular tachycardia (VT); predict that a multi-shock therapy is indicated by analyzing the time period; and in response to predicting that the multi-shock therapy is indicated, cause administration of the multi-shock therapy to the subject, the multi-shock therapy including a second electrical shock and a third electrical shock.
14. The medical device of clause 13, wherein the processor is configured to determine, by analyzing the ECG of the subject, the time period between the administration of the first electrical shock to the subject and the subsequent initiation of the shockable arrhythmia of the subject by: determining, by analyzing the ECG, a time at which the ECG of the subject transitions from into the shockable arrhythmia.
15. The medical device of clause 13 or 14, wherein the ECG is indicative of the shockable arrhythmia during a time period before administration of the first electrical shock.
16. The medical device of any of clauses 13 to 15, wherein the processor is configured to predict that the multi-shock therapy is indicated by analyzing the time period by: determining that the time period is less than a threshold time period.
17. The medical device of any of clauses 13 to 16, wherein the processor is configured to predict that the multi-shock therapy is indicated by: determining, by analyzing the ECG of the subject, a total amount of time in which the ECG is indicative of the shockable arrhythmia; and determining that the total amount of time in which the ECG is indicative of shockable arrhythmia is above a threshold.
18. The medical device of any of clauses 13 to 17, wherein the shockable arrhythmia includes the VF and the processor is configured to predict that the multi-shock therapy is indicated by: determining that an amplitude or amplitude spectrum area (AMSA) of the VF exhibited by the ECG is above a first threshold; or determining that the amplitude or AMSA of the VF exhibited by the ECG is below a second threshold.
19. The medical device of any of clauses 13 to 18, wherein the processor is configured to predict that the multi-shock therapy is indicated by: determining that the ECG is indicative of OMI or STEMI.
20. The medical device of any of clauses 13 to 19, wherein the processor is configured to cause administration of the multi-shock therapy to the subject by: causing administration of the first electrical shock along a first vector; and causing administration of the second electrical shock along a second vector or the first vector, the second vector being different than the first vector.
21. A first external defibrillator, including: a measurement circuit configured to detect an electrocardiogram (ECG) of a subject; a treatment circuit configured to output a first electrical shock to the subject during a first time period; and a processor configured to: determine, by analyzing the ECG of the subject: an amount of time that the ECG is indicative of ventricular fibrillation (VF); and an amount of time that the ECG is indicative of a non-VF heart rhythm; determine a VF score by dividing the amount of time that the ECG is indicative of the VF by the amount of time that the ECG is indicative of the non-VF heart rhythm; predict that a multi-shock therapy is indicated by determining that the VF score is greater than a threshold; and in response to predicting that the multi-shock therapy is indicated: outputting an instruction to administer a second electrical shock to the subject during a second time period, the second time period overlapping the first time period; and causing the treatment circuit to output the first electrical shock to the subject during the first time period.
22. The first external defibrillator of clause 21, wherein the processor is configured to determine the VF score further as a function of: an amplitude of the VF; an amplitude spectrum area (AMSA) of the VF; a presence of ST elevation in the ECG; and a presence of ST depression in the ECG.
23. The first external defibrillator of clause 21 or 22, further including: an output device configured to output the instruction to administer the second electrical shock to the subject during the second time period to a user; or a transceiver configured to output the instruction to administer the second electrical shock to the subject during the second time period to a second external defibrillator.
24. A method, including: determining an amount of time that an electrocardiogram (ECG) of a subject is indicative of a shockable arrhythmia, the shockable arrhythmia including ventricular fibrillation (VF) or ventricular tachycardia (VT); predicting that a multi-shock therapy is indicated by analyzing the amount of time; and in response to predicting that the multi-shock therapy is indicated, causing administration of the multi-shock therapy to the subject, the multi-shock therapy including a first electrical shock and a second electrical shock.
25. The method of clause 24, wherein the amount of time that the ECG of the subject is indicative of the shockable arrhythmia includes a time at which a third electrical shock is administered to the subject.
26. The method of clause 24 or 25, wherein the amount of time that the ECG of the subject is indicative of the shockable arrhythmia includes a sum of multiple time periods in which the ECG of the subject is indicative of the shockable arrhythmia, the multiple time periods being separated by a time period in which the ECG of the subject is indicative of a heart rhythm that is not the shockable arrhythmia.
27. The method of any of clauses 24 to 26, wherein predicting that the multi-shock therapy is indicated by analyzing the amount of time includes: determining a score by analyzing the amount of time; and comparing the score to a threshold.
28. The method of clause 27, wherein the score is proportional to the amount of time, and wherein comparing the score to the threshold includes determining that the score exceeds the threshold.
29. The method of clause 27 or 28, wherein determining the score is further dependent on: an AMSA of the ECG of the subject; or an amplitude of the ECG of the subject.
30. The method of any of clauses 27 to 29, wherein determining the score further includes: determining whether the ECG of the subject is indicative of STEMI or OMI.
31. The method of any of clauses 24 to 30, wherein the first electrical shock and the second electrical shock are administered by a single external defibrillator, the first electrical shock and the second electrical shock being temporally overlapping.
32. The method of any of clauses 24 to 31, wherein the first electrical shock is administered by a first external defibrillator and the second electrical shock are administered by a second external defibrillator, the first electrical shock and the second electrical shock being temporally overlapping.
33. A medical device, including: a processor configured to: determine an amount of time that an electrocardiogram (ECG) of a subject is indicative of a shockable arrhythmia, the shockable arrhythmia including ventricular fibrillation (VF) or ventricular tachycardia (VT); predict that a multi-shock therapy is indicated by analyzing the amount of time; and in response to predicting that the multi-shock therapy is indicated, cause administration of the multi-shock therapy to the subject, the multi-shock therapy including a first electrical shock and a second electrical shock.
34. The medical device of clause 33, wherein the amount of time that the ECG of the subject is indicative of the shockable arrhythmia includes a time at which a third electrical shock is administered to the subject.
35. The medical device of clause 33 or 34, wherein the amount of time that the ECG of the subject is indicative of the shockable arrhythmia includes a sum of multiple time periods in which the ECG of the subject is indicative of the shockable arrhythmia, the multiple time periods being separated by a time period in which the ECG of the subject is indicative of a heart rhythm that is not the shockable arrhythmia.
36. The medical device of any of clauses 33 to 35, wherein the processor is configured to predict that the multi-shock therapy is indicated by analyzing the amount of time by: determining a score by analyzing the amount of time; and comparing the score to a threshold.
37. The medical device of clause 36, wherein the score is proportional to the amount of time, and wherein the processor is configured to compare the score to the threshold by determining that the score exceeds the threshold.
38. The medical device of clause 36 or 37, wherein the processor is configured to determine the score by analyzing: an amplitude spectrum area (AMSA) of the ECG of the subject; or an amplitude of the ECG of the subject.
39. The medical device of any of clauses 36 to 38, wherein determining the score further includes: determining whether the ECG of the subject is indicative of STEMI or OMI.
40. The medical device of any of clauses 34 to 39, further including: a treatment circuit configured to output the first electrical shock to electrodes disposed on skin of the subject.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A first external defibrillator, comprising:
a measurement circuit configured to detect an electrocardiogram (ECG) of a subject;
a treatment circuit configured to output a first electrical shock to the subject during a first time period and to output a second electrical shock to the subject during a second time period; and
a processor configured to:
determine, by analyzing the ECG of the subject:
a time delay between administration of the first electrical shock and initiation of ventricular fibrillation (VF) in the ECG of the subject; and
a fibrillation time period during which the ECG is indicative of the VF;
determine a VF score as a function of the time delay and the fibrillation time period;
predict that a multi-shock therapy is indicated by comparing the VF score to a threshold; and
in response to predicting that the multi-shock therapy is indicated:
outputting an instruction to administer a third electrical shock to the subject during a third time period, the third time period overlapping the second time period; and
causing the treatment circuit to output the second electrical shock to the subject during the second time period.

2. The first external defibrillator of claim 1, wherein the processor is configured to determine the VF score further as a function of:
an amplitude spectrum area (AMSA) of the VF;
an amplitude of the VF;
a presence of ST elevation in the ECG; and
a presence of ST depression in the ECG.

3. The first external defibrillator of claim 1, further comprising:
an output device configured to output the instruction to administer the third electrical shock to the subject during the third time period to a user; or
a transceiver configured to output the instruction to administer the third electrical shock to the subject during the third time period to a second external defibrillator.

4. A method, comprising:
determining, by analyzing an electrocardiogram (ECG) of a subject, a time period between administration of a first electrical shock to the subject and a subsequent initiation of a shockable arrhythmia of the subject, the shockable arrhythmia comprising ventricular fibrillation (VF) or ventricular tachycardia (VT);
predicting that a multi-shock therapy is indicated by analyzing the time period; and
in response to predicting that the multi-shock therapy is indicated, causing administration of the multi-shock therapy to the subject, the multi-shock therapy comprising a second electrical shock and a third electrical shock.

5. The method of claim 4, wherein determining, by analyzing the ECG of the subject, the time period between the administration of the first electrical shock to the subject and the subsequent initiation of the shockable arrhythmia of the subject comprises:
determining, by analyzing the ECG, a time at which the ECG of the subject transitions into the shockable arrhythmia.

6. The method of claim 4, wherein the ECG is indicative of the shockable arrhythmia during a time period before administration of the first electrical shock.

7. The method of claim 4, wherein predicting that the multi-shock therapy is indicated by analyzing the time period comprises:
determining that the time period is less than a threshold time period.

8. The method of claim 4, wherein predicting that the multi-shock therapy is indicated further comprises:
determining, by analyzing the ECG of the subject, a total amount of time in which the ECG is indicative of the shockable arrhythmia; and
determining that the total amount of time in which the ECG is indicative of the shockable arrhythmia is above a threshold.

9. The method of claim 4, wherein the shockable arrhythmia comprises the VF and predicting that the multi-shock therapy is indicated further comprises:
determining that an amplitude of the VF exhibited by the ECG is above a threshold.

10. The method of claim 4, wherein the shockable arrhythmia comprises the VF predicting that the multi-shock therapy is indicated further comprises:
determining that an amplitude of the VF exhibited by the ECG is below a threshold.

11. The method of claim 4, wherein predicting that the multi-shock therapy is indicated further comprises:
determining that the ECG is indicative of OMI or STEMI.

12. The method of claim 4, wherein causing administration of the multi-shock therapy to the subject comprises:
causing administration of the first electrical shock along a first vector; and
causing administration of the second electrical shock along the first vector or a second vector, the second vector being different than the first vector.

13. A medical device, comprising:
a measurement circuit configured to detect an electrocardiogram (ECG) of a subject; and
a processor configured to:
determine, by analyzing the ECG of the subject, a time period between administration of a first electrical shock to the subject and a subsequent initiation of a shockable arrhythmia of the subject, the shockable arrhythmia comprising ventricular fibrillation (VF) or ventricular tachycardia (VT);
predict that a multi-shock therapy is indicated by analyzing the time period; and
in response to predicting that the multi-shock therapy is indicated, cause administration of the multi-shock therapy to the subject, the multi-shock therapy comprising a second electrical shock and a third electrical shock.

14. The medical device of claim 13, wherein the processor is configured to determine, by analyzing the ECG of the subject, the time period between the administration of the first electrical shock to the subject and the subsequent initiation of the shockable arrhythmia of the subject by:
determining, by analyzing the ECG, a time at which the ECG of the subject transitions from into the shockable arrhythmia.

15. The medical device of claim 13, wherein the ECG is indicative of the shockable arrhythmia during a time period before administration of the first electrical shock.

16. The medical device of claim 13, wherein the processor is configured to predict that the multi-shock therapy is indicated by analyzing the time period by:
determining that the time period is less than a threshold time period.

17. The medical device of claim 13, wherein the processor is configured to predict that the multi-shock therapy is indicated by:
determining, by analyzing the ECG of the subject, a total amount of time in which the ECG is indicative of the shockable arrhythmia; and
determining that the total amount of time in which the ECG is indicative of shockable arrhythmia is above a threshold.

18. The medical device of claim 13, wherein the shockable arrhythmia comprises the VF and the processor is configured to predict that the multi-shock therapy is indicated by:
determining that an amplitude or amplitude spectrum area (AMSA) of the VF exhibited by the ECG is above a first threshold; or
determining that the amplitude or AMSA of the VF exhibited by the ECG is below a second threshold.

19. The medical device of claim 13, wherein the processor is configured to predict that the multi-shock therapy is indicated by:
determining that the ECG is indicative of OMI or STEMI.

20. The medical device of claim 13, wherein the processor is configured to cause administration of the multi-shock therapy to the subject by:
causing administration of the first electrical shock along a first vector; and
causing administration of the second electrical shock along a second vector or the first vector, the second vector being different than the first vector.
